(19) United States
(12) Patent Application Publication
(10) Pub. No.: US 2007/0078492 A1
(43) Pub. Date: Apr. 5, 2007

(54) Method and device to convert cardiac and other body movements into electricity to power any implantable medical system

(76) Inventors: Piergiorgio Tozzi, Lausanne (CH); Ludwig Karl Von Segesser, Lausanne (CH)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/240,459

(22) Filed: Oct. 3, 2005

(51) Int. Cl. A61N 1/00

(52) U.S. Cl. 607/35

(57) ABSTRACT

Method and device to convert movement of human body part such as heart, diaphragm, skeletal muscles, etc. into electrical energy using smart materials like electroactive polymers, piezoelectric materials or other materials which passive deformation produces electric signals.

### BACKGROUND OF THE INVENTION

Any medical device implanted in the human body that requires electric energy to work has two potential source of energy: battery pack or external energy source connected to the electric network.

The battery pack can be implanted under the skin, as in pacemaker, defibrillator, neurostimulator, but has limited lifetime and has to be replaced after months or years. The battery replacement requires always a surgical procedure. In few cases, like totally implantable cardiac assist device (LionHeart), the battery can be recharged with transcutaneous energy transfer mean, but, in this case, battery autonomy is less than 60 minutes. Therefore, the rechargeable implantable battery is constantly powered by an extracorporeal power source.

The external energy source is used in almost all cardiac assist devices: basically, the medical device is implanted into the human body and has tubes piercing the patient skin to plug in the electric network. This system increases the risk of infections and requires patients to be constantly tethered to an external power source.

Therefore, each of the existing solutions to power an implantable medical device has a specific limitation. There is a clear need of an alternative way to power any implantable medical device to avoid surgical procedures to replace batteries and the need to be constantly tethered to an external power source.

### SUMMARY OF THE INVENTION

The present invention relates to a novel method and device aimed to overcome the above identified prior art drawbacks.

This invention refers to a method to convert the natural movement of the heart and other muscles like diaphragm and skeletal muscles—that is kinetic energy—into electrical energy using smart materials like electroactive polymers, piezoelectric materials or other materials with “piezoelectric_behaviour”, where “piezoelectric behaviour” means that passive deformation produces electric signals.

### BRIEF DESCRIPTION OF THE DRAWINGS

The technical features of the present invention, in accordance with the above-mentioned aims, are set out in the claims herein and the advantages more clearly illustrated in the detailed description which follows, with reference to the accompanying drawings, which illustrate a preferred embodiment without limiting the scope of application, and in which:

FIG. 1 schematically shows a device of the invention applied to a cardiac contraction assist device;

FIG. 2: shows a possible arrangement of smart material according to the invention for electric signal amplification in serial (A), parallel (B) or combined (C), in one or several layers or other orientations.

### DESCRIPTION OF THE PREFERRED EMBODIMENTS

In an exemplary embodiment of the invention an electroactive polymer (or EAP) strip is applied to the external surface of the heart in such a way that heart movements—that is contraction and relaxation—causes passive movement of the strip.

When the strip size is of 5×15×0, 5 mm and it is bended of 45° or more, it generates a 10 to 90 millivolt electric signal at its ends.

Accordingly, the heart movement causes the piezoelectric material to bend and this bending generates an electric signal.

The electric signal generated is amplified and made available to further uses.

A typical application for the present design is to power or recharge the battery of any implantable medical device (e.g. pace-makers, glucose detectors, recording devices, defibrillators, etc).

Further developments include the power source of an artificial heart, ventricular or atrial assist devices of traditional design or based on artificial muscles, as well as any other implant requiring electrical power.

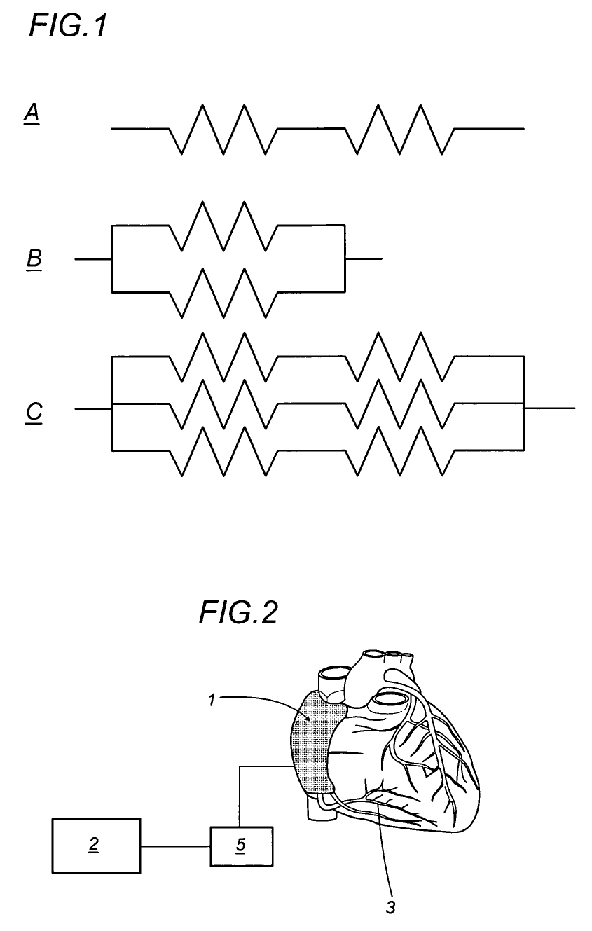

Advantageously, with reference to FIG. 1, the implantable device may consist of an artificial tissue 1, comprising electroactive elements.

In this case, the mechanical energy available at a first region of the body and converted by a device 2, can be transmitted to the medical device 1 (preferably through a control unit 5) to be used to assist the contraction of a second part of the body, for example an heart region 3.

In addition, in the latter case both the power generating device 5 and the medical powered device 1 do not comprise and external energy source, so making safer the whole implantation.

Finally, smart materials can be placed between any other moving parts within the body in order to convert the movement into electric energy.

More particularly, the invention consists of attaching by suturing, gluing or other convenient methods, smart material on the heart surface or other parties of the body.

Advantageously, in order to maximize the electric signal generated by the device, the device may be shaped in form of one or more strips or webs, which can be arranged according to the main contraction lines of the part of the body supporting the device.

Furthermore, the electric signal generated can be amplified in serial (A), parallel (B) or combined (C), in one or several layers or other orientations (FIG. 2) in order to accomplish different needs.

The invention described may be used for evident industrial applications and can be subject to numerous modifications and variations without thereby departing from the scope of the inventive concept. Moreover, all the details of the invention may be substituted by technically equivalent elements.

What is claimed is:

1) A method to convert a body movement into electricity, comprising the steps of:
providing a three dimensional web consisting of a plurality of elements generating electric signals when subject to passive deformation;
fix the web to a contractile region of the body of a patient; the elements being spatially arranged according to at least one direction of contraction of the body region;
supply the generated electric signals to power an implantable medical device.

2) A method according to claim 1, wherein said generated electric signal is amplified before being supplied to said implantable device

3) A method according to claim 1, wherein said body region is a cardiac region.

4) A method according to claim 1, wherein said web is sutured to the body region.

5) A method according to claim 1, wherein said web is glued to the body region.

6) A device to convert a body movement into electricity, comprising:
a three dimensional web consisting of a plurality of elements generating electric signals when subject to passive deformation, the elements being spatially arranged according to at least one direction of contraction of the body region;
means to fix the web to a contractile region of the body of a patient;
means to supply the generated electric signals to an implantable medical device.

7) A device according to claim 6, wherein said element generating electric signals comprise a strip of an electroactive polymers (EAP).

8) A medical implantable device comprising a power source comprising an electric signal generating device comprising:
a three dimensional web consisting of a plurality of elements generating electric signals when subject to passive deformation, the elements being spatially arranged according to at least one direction of contraction of the body region;
means to fix the web to a contractile region of the body of a patient;
means to supply the generated electric signals to an implantable medical device.

9) An implantable device according to claim 8, wherein said element generating electric signals comprise a strip of an electroactive polymers (EAP).

10) An implantable device according to claim 8, wherein the implantable device is a cardiac assist device comprising an artificial tissue made of electroactive elements capable of contract when subject to an electric signal.
